(19) United States
(12) Patent Application Publication
(10) Pub. No.: US 2022/0396680 A1
(43) Pub. Date: Dec. 15, 2022

(54) Method of Separating a First Contaminant from a Feed Stream and Reactor System for Carrying Out the Method

(71) Applicant: Ioniqa Technologies B.V., Eindhoven (NL)

(72) Inventors: Joost Robert WOLTERS, Waalre (NL); Michael Josef DE GROOT, Dordrecht (NL); Jan Volkert ZANDER, Haarlem (NL)

(73) Assignee: Ioniqa Technologies B.V., Eindhoven (NL)

(21) Appl. No.: 17/775,138

(22) PCT Filed: Nov. 6, 2020
(86) PCT No.: PCT/EP2020/081332

§ 371 Date: May 6, 2022

(30) Foreign Application Priority Data
Nov. 7, 2019 (NL) 2024181

(51) Int. Cl. C08J 11/24; B01D 21/26; B01D 21/00; B01D 21/34; B01D 11/04

(52) U.S. Cl. CPC C08J 11/24; B01D 21/267; B01D 21/0084; B01D 21/34; B01D 11/043; C08J 2331/08; B01D 2257/7022

(57) ABSTRACT

The invention relates to a method of separating a first contaminant from a feed stream further comprising a condensation polymer. The invention further relates to a reactor system for carrying out the method, comprising at least one depolymerization vessel, configured for depolymerizing a condensation polymer into monomer, dimer, trimer and/or oligomer, which depolymerizing occurs in an alcoholic solvent, wherein said condensation polymer is provided as a feed stream further comprising a first contaminant, the reactor system comprising a separation stage, said separation stage comprising a separation vessel, downstream of the depolymerization vessel, configured for collecting a first contaminant, wherein said first contaminant is separated from the alcoholic solvent on the basis of a density separation so that the first contaminant is arranged on top of the alcoholic solvent.

### CROSS-REFERENCE TO RELATED APPLICATION

This application claims the benefit of the filing date of International Patent Application No. PCT/EP2020/081332 filed Nov. 6, 2020, entitled, “Reactor System And Method Of Separating A First Contaminant From A Feed Stream” and Netherland Application No. NL2024181 filed Nov. 7, 2019, entitled, “Reactor System And Method Of Separating A First Contaminant From A Feed Stream”, both of which are hereby incorporated by reference as if fully set forth herein.

### FIELD OF THE INVENTION

The invention relates to a method of separating a first contaminant from a feed stream further comprising a condensation polymer.

The invention further relates to a method of processing a feed stream comprising a condensation polymer and a first contaminant so as to obtain purified monomer and/or purified dimer.

The invention also relates to a reactor system for carrying out the method. The reactor system comprises at least one depolymerization vessel configured for depolymerizing a condensation polymer into monomer, dimer, trimer and/or oligomer in an alcoholic solvent, wherein said condensation polymer is provided as part of a feed stream further comprising a first contaminant.

### BACKGROUND OF THE INVENTION

It has been recognized that recycling of polymers in waste material is necessary, so as to prevent huge landfills and so as to make efficient use of raw materials. Polymers are used in a large variety in packaging, construction materials, textile and so on. Polymers are generally subdivided into polymers obtained by radical polymerization and condensation polymers. The first group includes well-known members as polyolefins (such as polyethylene and polypropylene) and polyvinylchloride. The second group includes polyesters, polyamides, polyethers and polyurethanes. Well-known polyesters include polyethylene terephthalate (PET), polybutylene succinate and polylactic acid (PLA). Well-known polyamides include nylon-6 and nylon-6,6.

Packaging waste comprising a variety of bottles is nowadays collected separately and thereafter sorted in a pre-sorting and typically processed to flakes or other pieces with sufficiently small volume. The sorting herein is for instance carried out by optical recognition, based on information that a specific bottle is made of a certain material. As a consequence, it has become feasible to provide feed streams that largely comprise one or two types of polymer, such as polyethylene, polypropylene or PET. A feed stream can then be provided for processing into new raw material of specific quality. For polyolefins, such processing involves cleaning, sorting and mixing to specific product grades. For condensation polymers, such processing involves depolymerization into monomer and the like.

It is known that the quality of the resulting circular raw material strongly depends on the removal of contaminants. These contaminants include pigments and other additives such as fillers and plasticizers that may be present in the polymer material. These contaminants further include other mostly polymer material which could not be removed in the pre-sorting. Since the waste material tends to come from a variety of sources, even when being consumer packaging waste, still there is a significant unpredictability as to the amount of contaminants and the type of contaminants, e.g. depending on the source, part of the season and or on a batch-to-batch basis.

One way of dealing therewith is the performing of extensive cleaning and sorting of the feed, e.g. with water. However, it would give rise to significant costs for condensation polymers. After such thorough cleaning and sorting, condensation polymers still need to be depolymerized into monomers, dimers, oligomers and the like with sufficient yield. The useful raw material, typically the monomer, is then to be collected and crystallized. This raw material needs to be cleaned thoroughly, for instance by filtration, treatment with activated carbon and/or ion exchange resins. Overall, the total costs of cleaning and sorting the feed and subsequent depolymerization and purification of the monomer would render the entire process too expensive, because they require a factory on its own.

Furthermore, it has been observed that the depolymerization by means of glycolysis is very sensitive to the presence of water in the depolymerization reactor. The presence of minor amounts of water originating from the prior cleaning steps already results in the formation of significant amounts of unwanted by-products. It is cumbersome to separate such by-products out, as they will dissolve well into the used solvents. However, such by-products have a negative effect on the quality of polymer material obtained from a subsequent polymerization into a new polymer.

A method of separating a contaminant, such as a colorant, from a feed stream comprising a condensation polymer is disclosed in US 2006/0074136A1. Colored polyester is depolymerized by the addition of glycol to form the monomer bis hydroxyethyl terephthalate (BHET). The formed BHET is first contacted with activated carbon to remove the colorant, and the colorant is then further extracted to produce purified BHET. The purified BHET may be separated from the extraction solvent by decanting or centrifuging. Polymers such as polyvinyl chloride having a higher density than the depolymerized condensation polymer from the feed stream may be removed from the depolymerized reaction mixture by filtering. The method of US 2006/0074136A1 however is not adequate to remove contaminants with a density lower than the depolymerized condensation polymer from the feed stream. The applied filter is not selective enough and first contaminant remaining in the depolymerized reaction mixture may end up in the activated carbon bed and provide problems such as clogging and the like.

EP 1914270A1 also discloses a method of separating a contaminant, such as a dye, from a feed stream comprising a condensation polymer. The dye is extracted with a xylene extracting solvent and an alkylene extracting solvent in combination. EP 1914270A1 discusses that fibers of polymers having a relatively small specific density may be separated from the feed stream in the reactor vessel by floating onto the liquid surface of the depolymerized solution in the reactor vessel itself, and subsequent extraction. Although this method is easy, the polymer may still not be removed adequately.

### SUMMARY OF THE INVENTION

It is therefore an object of the invention to provide a method of separating at least one contaminant with a density lower than the depolymerized condensation polymer from a stream further comprising the condensation polymer suitable for depolymerization under reaction conditions which can be carried out in a suitable reactor system.

It is another object of the invention to provide a reactor system for depolymerizing a condensation polymer suitable for depolymerization under reaction conditions, in a stream further comprising a solvent and at least one contaminant, into monomer, dimer, trimer and/or oligomer in a more economically feasible way.

The invention has more in particular the object to provide a method which is able to separate the stream in a way not requiring extensive cleaning and/or sorting which may involve the use of water.

This object is achieved with a method according to claim 1. The method of separating a first contaminant from a feed stream further comprising a condensation polymer comprises the steps of:
supplying the feed stream, an alcoholic solvent and optionally a depolymerization catalyst into a depolymerization vessel and mixing thereof to form a reaction mixture;
depolymerizing at least a portion of said condensation polymer in said reaction mixture into monomer, dimer, trimer and/or oligomer under said reaction conditions;
transferring the reaction mixture after the depolymerization of at least a portion of said condensation polymer in said reaction mixture to a separation stage comprising a separation vessel,
collecting the first contaminant, wherein said first contaminant is separated from the alcoholic solvent on the basis of a density separation in the separation stage, in particular so that first contaminant is arranged on top of the alcoholic solvent in the separation vessel;
further comprising the step of cooling the reaction mixture with cooling means before the collecting step to ensure that the reaction mixture in the separation vessel is at a lower temperature than the depolymerization vessel such that the first contaminant that is liquid and/or dissolved in the depolymerization vessel at least partially precipitates and the cooled reaction mixture separates into a main phase that comprises the reaction products from the depolymerization and the alcoholic solvent, and a first contaminant phase in the form of solid particles and/or a solid layer.

The method is suitably carried out in a reactor system comprising at least one depolymerization vessel, configured for depolymerizing a condensation polymer into monomer, dimer, trimer and/or oligomer, which depolymerizing occurs in an alcoholic solvent, wherein said condensation polymer is provided as a feed stream further comprising a first contaminant, wherein said reactor system further comprises a separation stage, downstream of the depolymerization vessel, said separation stage comprising a separation vessel, having an inlet for introducing the reaction mixture originating from the depolymerization vessel into the separation vessel, configured for collecting the first contaminant, in which separation vessel said first contaminant is separated from the alcoholic solvent on the basis of a density separation so that first contaminant is arranged on top of the alcoholic solvent, wherein the reactor system further comprises a cooling means for ensuring that the separation vessel is at a lower temperature than the depolymerization vessel such that the first contaminant that is liquid and/or dissolved in the depolymerization vessel at least partially precipitates and the cooled reaction mixture separates into a main phase that comprises the reaction products from the depolymerization and the alcoholic solvent, and a first contaminant phase in the form of solid particles and/or a solid layer.

It has surprisingly been found that it is possible to, by depolymerizing the condensation polymer in the feed stream into monomer, dimer, trimer and/or oligomer, remove contaminants which have a density lower than said depolymerized condensation polymer from the feed stream.

These contaminants have an ability to form a phase separate from the reaction products from the depolymerization of condensation polymer which will float on the solvent. These contaminants are hereafter referred to—collectively— as the first contaminant.

The first contaminant is liquid during the depolymerization step and/or is at least partly dissolved during the depolymerization step in the alcoholic solvent. The reaction mixture transferred from the depolymerization vessel to the separation stage comprises the alcohol solvent, the monomer, dimer, trimer and/or oligomer obtained from the condensation polymer and the first contaminant.

The reactor system further comprises a cooling means for cooling the reaction mixture such that the separation vessel is at a lower temperature than the depolymerization vessel and that the first contaminant that is liquid and/or dissolved in the depolymerization vessel at least partially precipitates. As a result, in the separation stage the reaction mixture, e.g. the contents of the separation vessel, easily separates into a main phase and at least a first contaminant phase. The first contaminant of the first contaminant phase, when at least partially precipitated, forms solid particles and/or a solid layer. The formation of the solid particles and/or the solid layer facilitates an easy separation of the first contaminant phase from the main phase and handling of the first contaminant. The first contaminant has a density lower than said depolymerized condensation polymer.

According to the invention, the cooling means is configured for cooling the first contaminant and the alcohol solvent such that the first contaminant at least partially precipitates.

In particular, the method comprises the step of cooling the reaction mixture with cooling means to ensure that the reaction mixture in the separation vessel is at a lower temperature than the depolymerization vessel such that the first contaminant that is liquid and/or dissolved in the depolymerization vessel at least partially precipitates.

In accordance with the invention, the provision of a separation stage, said separation stage comprising a separation vessel, configured for collecting of the first contaminant, arranged downstream of the depolymerization vessel facilitates easy collection of the first contaminant, i.e., from the main phase. As a consequence, it is no longer required to clean and sort the stream before depolymerization, substantially reducing the chance of the aforementioned by-product formation. The condensation polymer may be completely depolymerized before transfer of the reaction mixture to the separation vessel, but it may also be just substantially depolymerized, i.e. the conversion may also be lower, for instance no more than 90%, no more than 80%, no more than 70% or even no more than 60% on a stoichiometric basis, or even less than that.

The depolymerization vessel preferably comprises a first inlet for introduction of the feed stream, and a further inlet for the alcoholic solvent. A depolymerization catalyst may be provided separately and/or as part of the alcoholic solvent. The feed stream may further comprise alcoholic solvent, which alcoholic solvent will primarily act as a carrier liquid for the feed stream that is otherwise typically in solid form. The depolymerization vessel typically comprises a mixer to mix the reaction mixture during depolymerization. In general, any vessel suitable for depolymerization of a condensation polymer may be selected as the depolymerization vessel. Rather than a single depolymerization vessel a plurality of serial and/or parallel depolymerization vessels may be present, which may have one or more feedback loops. The depolymerization vessel is suitably configured for a volume in the range of 0.1-100 m3, such as 10-50 m3. This is deemed sufficient to enable a feed flow rate in the order of 10-100 kton/year.

The separation stage comprises the separation vessel and may comprise additional separation means for separating the main phase and at least a first contaminant phase from each other. The additional separations means may comprise a sieve bend unit and/or a cyclone and/or any other separations means for separating the main phase and at least a first contaminant phase from each other.

The separation vessel preferably comprises an inlet to introduce the reaction mixture originating from the depolymerization vessel into the separation vessel. The separation vessel preferably comprises an outlet for carrying off the main phase after separation. This may allow the separation in the separation vessel to be carried out in a continuous manner, although batch processing still a possibility with such an outlet. When the separation vessel comprises both said inlet and said outlet, said inlet and said outlet are preferably spaced apart from each other, in order to obtain a residence time for the contents of the separation vessel allowing the aforementioned separation to occur. Said inlet and said outlet are preferably spaced apart in a direction which is parallel to the bottom of the separation vessel. The space which is thereby created increases the residence time in the separation vessel, providing more time for the first contaminant to constitute a phase separate from the main phase. It is preferred to bring or keep the separation vessel at a lower temperature than the depolymerization vessel, since it is observed that a lower temperature leads to an increased chance of phase separation between the depolymerized condensation polymer and the first contaminant.

In order to be able to collect the majority of the first contaminant, the first contaminant phase may also comprise other constituents than just the first contaminant. A first contaminant phase which comprises other constituents is preferably passed through a separating means, e.g. a membrane or sieve, which is selected to separate the first contaminant phase into a first phase which in particularly comprises the first contaminant, and a second phase which in particularly comprises the other constituents of the first contaminant phase. The second phase is then preferably recycled, e.g. to the separation vessel and/or the depolymerization vessel. Examples of other constituents include alcoholic solvent, monomer, dimer, trimer, oligomer and condensation polymer dissolved and/or dispersed in the alcoholic solvent, any co-solvent (such as water or an aqueous solution), catalyst and further contaminants that are either dissolved or dispersed in the alcoholic solvent. When using a top outlet, the first contaminant will preferably constitute between 5 and 95 wt % of the first contaminant phase, preferably from 8-60 wt %, or from 10-40 wt %. The other major constituent of the first contaminant phase is typically the alcoholic solvent. To the extent that the other constituents are dispersed in the alcoholic solvents, these will be of a size and density such that they do not move downwards through the alcoholic solvent.

Additionally or alternatively, the separation vessel may comprise a main outlet for carrying off the whole contents of the separation vessel, including both a main phase and a first contaminant phase when present. The outlet may be used to further process the contents of the separation vessel downstream of the separation vessel to separate the main phase and the first contaminant phase from one another downstream of the separation vessel, such as by using a sieve bend unit and/or a cyclone according to the invention.

The separation stage may further comprise a sieve bend unit arranged downstream of the separation vessel for separating the first contaminant from a filtrate stream comprising the alcoholic solvent via an inclined screen. The filtrate stream may in particularly comprises other constituents than the first contaminant. The sieve bend unit may be coupled to the main outlet of the separation vessel for receiving the whole contents of the separation vessel or may be coupled to the top outlet of the separation vessel for receiving the first contaminant phase. The sieve bend unit comprises a sieve or screen to separate the solid first contaminant part from liquid parts. The sieve or screen is preferably arranged inclined to allow the residue comprising the solid first contaminant part to slide downwards along the inclined sieve bend towards a storage vessel. The storage vessel is arranged for storing the solid first contaminant part, which falls due to gravity into the storage vessel.

In particular, the filtrate stream obtained from the sieve bend device may be arranged to be at least partly recirculated to the separation vessel.

Additionally or alternatively, the separation stage may comprise at least one cyclone device arranged downstream of the depolymerization vessel for separating a low density stream comprising the first contaminant from a high density stream comprising the alcoholic solvent on the basis of a density separation. Said at least one cyclone device may be arranged downstream of the separation vessel. The at least one cyclone device may be coupled to the main outlet of the separation vessel for receiving the whole contents of the separation vessel or may be coupled to the top outlet of the separation vessel for receiving the first contaminant phase.

Additionally or alternatively, one or more cyclone devices of said at least one cyclone device may be arranged upstream of the separation vessel.

In particular, a filter device may be arranged for receiving at least one low density stream from said at least one cyclone device for filtering the first contaminant from the alcoholic solvent.

### (Separation Vessel)

Preferably, the separation vessel comprises a bottom with an upright circumferential wall, defining a space for the separation of the contents of the separation vessel to occur. The vessel may be closed and be provided with one or more manholes, or have an open top.

The problem of contaminant separation is more profoundly present in the treatment of waste. Therefore, the feed stream preferably is a waste stream. In many waste streams, an amount of contaminants is rather unpredictable and may depend on supplier of the waste stream, and waste collection parameters, such as season, type of polymer waste, for instance packages, country and/or location. The contaminants are typically part of a mixture. It is furthermore feasible that part of the contaminants is included within the condensation polymer. Such contaminants are however most frequently soluble in the alcoholic solvent and/or would rather become part of a separated phase that is heavier than the alcoholic solvent. Preferably, the condensation polymer comprises a polyester such as polyethylene terephthalate, and the first contaminant comprises a polyolefin, such as polyethylene or polypropylene. The polyolefins, which will typically be present in a concentration of between 0 and 5 percent by weight of the stream, will form a floating and typically solid layer on top of the substantially depolymerized condensation polymer phase, which is easily collected with a suitably configured separation vessel. Polyolefins typically form non-sticky globular particles, which are collected easily with the separation vessel of the reactor system according to the invention. When the first contaminant comprises polyolefin, the weight percentage of the first contaminant in the first contaminant phase is typically between 30 and 50 percent by weight, but may even be equal to or lower than 10 percent by weight. After collection, the polyolefins may be post-processed for reuse.

The remaining main phase after the separation from the first contaminant and possibly a second contaminant, which is hereafter discussed in more detail, which main phase comprises the reaction products from the depolymerization, may be transferred to a suitable post-processing unit after exiting the separation vessel. The post-processing unit for post-processing the main phase may for instance comprise at least one of a centrifuge, a unit loaded with active carbon and/or a crystallization unit.

In a preferred embodiment, the separation vessel comprises a top outlet, configured for collecting the first contaminant.

The top outlet is an outlet arranged at a location for contacting the contents of the separation vessel. Furthermore, the top outlet is preferably located in the upper half of the separation vessel, or even the upper quarter of the separation vessel in order to utilize the volume of the separation vessel economically, also allowing the phase comprising at least the first contaminant to have a substantial volume or layer thickness. The top outlet may for instance be arranged in the top or ceiling of the separation vessel.

Solid layers are preferred, since these are in particular easy to be removed from the remaining reaction mixture, because of the difference in physical characteristics between said phases.

Apart from the preferred embodiments of the top outlet which are discussed hereafter, the top outlet may also be embodied as a simple elongate pipe with an inlet opening for collecting the first contaminant. This pipe may extend within the separation vessel in a vertical direction, from the inlet opening towards the bottom of the separation vessel. In the context of the invention, the top outlet is an outlet arranged at an upper side of the separation vessel, and configured to be present, in use of the separation vessel when at least partially filled with a mixture comprising a liquid, at or above a level of the liquid. When the top outlet is above the level of the liquid during normal use, means such as a valve in or at the outlet, are suitably available, so as to control the level of liquid to arrive at a level, during a period of time, at which the first contaminant and optionally any further constituents of the first contaminant phase may be removed.

In a further preferred embodiment, the top outlet comprises at least one of a skimmer or a scumming device.

A practical embodiment of the top outlet is a skimmer or a scum pipe. Skimmers and scum pipes are types of equipment known in the distant technical field of wastewater treatment, but have not yet been considered for the collection of contaminants out of a stream comprising condensation polymers and/or depolymerization products thereof, such as oligomer, trimer, dimer and monomer.

A skimmer may be defined as a receptacle with a bottom and an upright circumferential wall, of which the upper edge defines the inlet of the skimmer (and thereby the outlet of the separation vessel), for collecting the first contaminant. The skimmer may be a container which is, apart from the inlet, closed, but may also be connected to a discharge means, such as a drain pipe. The skimmer may for instance have the shape of a funnel.

A scumming device may be defined as an elongate device, which comprises an inlet or a multitude of inlets arranged next to each other extending substantially in an axial direction of the device (and thereby the outlet of the separation vessel), for collecting the first contaminant. The scumming device is typically oriented with its axial direction parallel to the surface of the reaction mixture in the separation vessel, and preferably perpendicular to the direction of flow between the inlet and the at least one outlet of the separation vessel. The scumming device may be a scum pipe, in which case the inlet extends over a part of the circumference of the pipe, e.g. less than 25% or even less than 20% of the circumference thereof. The scumming device typically is also connected to a discharge means, such as a drain pipe.

While it is typically sufficient to either provide a skimmer or a scumming device, it may also be preferred to provide both in the same separation vessel.

It is possible to have the position of the opening of the skimmer or scumming device at a fixed position, but in another embodiment, the position of the top outlet is adjustable.

As mentioned, feed streams comprising condensation polymers with contaminants may change, e.g. depending on the source, part of the season and/or on a batch-to-batch basis, process conditions may be subject to change. For instance, the quality and/or quantity of the contaminant in the feed stream may change within a feed stream or between feed streams, and the total flow (in volume per unit of time) may change. Such or other changes in process conditions may lead to variation in the location and/or the amount of the first contaminant in the separation vessel and in particular the distance of the first contaminant from the bottom of the separation vessel. Furthermore, it may be preferred to collect the first contaminant phase intermittently, allowing the first contaminant phase to grow to a certain thickness (as in size) before starting the collection with the top outlet. In order to accommodate for this, it is preferred to embody the top outlet, e.g. skimmer or scumming device, in an adjustable manner, e.g. either manually or electronically controlled. The adjustability may be restricted to a defined range which makes it unsuitable for collecting contents from the bottom of the separation vessel.

The top outlet may for instance be arranged, e.g. mounted, in the separation vessel by means of, e.g. fixed to, an adjustable frame, which allows the top outlet to be arranged at a plurality of different height levels with respect to the bottom of the separation vessel.

In the case of a scum pipe, the scum pipe is preferably rotatably mounted around its axis. Rotation of the scum pipe around its axis will change the distance from the inlet or inlets to the bottom of the separation vessel. This distance may thereby be varied over a specified range, without requiring the provision of a frame, which provides a relatively simple way of obtaining adjustability which is sufficient to accommodate for the most typical changes in process conditions.

According to the invention, the cooling means are configured such that the first contaminant that is liquid and/or dissolved in the depolymerization vessel forms a separate, solid, phase due to the at least partially precipitation of said first contaminant.

Formation of a separate first contaminant phase, wherein first contaminant is in solid state, allows the contaminant concerned to be collected more easily. The exact temperature required in order to obtain a separate, solid, phase depends on the contaminant and condensation polymer present, but will be readily apparent to a person skilled in the art.

According to the invention, the reactor system comprises a cooling means for ensuring that the separation vessel is at a lower temperature than the depolymerization vessel.

It is preferred to bring or keep the separation vessel at a lower temperature than the depolymerization vessel, since it is observed that a lower temperature leads to an increased chance of phase separation between the depolymerized condensation polymer and the first contaminant, which is observed in a pronounced way with polyolefins as the contaminant with polyethylene terephthalate as the condensation polymer in particular. For this purpose, a lower temperature may in this respect be defined as a temperature difference of at least 10° C., at least 30° C., at least 50° C., at least 70° C., at least 80° C., at least 90° C., at least 100° C., or even at least 110° C.

In particular, the cooling means is configured for cooling the first contaminant and the alcohol solvent such that the first contaminant at least partially precipitates. In particular, the method comprises the step of cooling the reaction mixture with cooling means to ensure that the reaction mixture in the separation vessel is at a lower temperature than the depolymerization vessel such that the first contaminant that is liquid and/or dissolved in the depolymerization vessel at least partially precipitates. The cooling may be achieved in a number of ways, e.g. by the provision of equipment, e.g. a heat exchanger, which is configured and/or suitable for cooling down the reaction mixture, or the introduction of a fluid into the reaction mixture and/or the separation vessel with a lower temperature than the reaction mixture. The cooling may be performed at various locations, which are preferably downstream of the depolymerization vessel, such as within the separation vessel or between the outlet of the depolymerization vessel and the inlet of the separation vessel.

In another preferred embodiment, the separation vessel is downstream of a water supply.

It has been observed that the addition of water leads to increased phase separation of the first contaminant. Furthermore, the water introduced may have a lower temperature than the reaction mixture in the depolymerization vessel, thereby cooling down the other constituents of the contents of the separation vessel, such as the reaction mixture originating from the depolymerization vessel. As a consequence, the water supply may therefore be one of the, or even the only cooling means of the reactor system.

In this respect, water may be defined to also encompass impure water and/or aqueous solutions, e.g. solutions which consist of more than 85 percent by weight of water, maybe even more than 90 percent by weight of water, or maybe even more than 95 percent by weight of water. It may be preferred to use a residual aqueous solution which may originate from another process outside the current reactor system.

In another embodiment, the reactor system comprises mixing means for mixing the contents of the separation vessel.

In the operation of the reactor system, it is highly preferred to ensure that the contents of the separation vessel, e.g. the reaction mixture originating from the depolymerization vessel and the water originating from the water supply, are mixed when or shortly after entering the separation vessel. This is in particular advantageous when carrying out a batch process. Mixing in this respect may also encompass the presence of a turbulent flow regime within the contents of the separation vessel, regardless of the cause of this regime. This allows for proper mixing of various constituents of the reaction mixture originating from the depolymerization vessel and the water originating from the water supply, thereby contributing to a more pronounced phase separation of the first contaminant.

The mixing means may be arranged in a supply line towards the inlet of the separation vessel, e.g. by the provision of an inline mixing means, for mixing the water from the water supply and the reaction mixture from the depolymerization vessel, before entry into the separation vessel. In other words, the supply lines to the separation vessel itself may then be regarded (part of) the mixing means, and it may not be necessary to arrange further mixing means within the separation vessel.

In addition to this, or as an alternative, the mixing means may however also be embodied in the separation vessel, close to any inlet of the separation vessel (e.g. the inlet connected to the depolymerization vessel and/or the inlet connected to the water supply), in order to be able to mix the contents of the separation vessel shortly after entry into the separation vessel, thereby efficiently using the space in the separation vessel to allow for phase separation. The mixing means are preferably embodied as a stirrer. The provision of a mixing means in the separation vessel is in particular beneficial in absence of mixing means in the supply line.

In a more preferred embodiment, the reactor system further comprises a pervious plate, arranged within the separation vessel downstream of and preferably adjacent to the mixing means. This pervious plate defines a mixing chamber in and/or upstream of the separation vessel and a settling chamber downstream of the mixing chamber.

The provision of a pervious plate, which may also be called a calming plate or a plate provided with a plurality of through-holes, allows the contents of the separation vessel to settle or calm down in the flotation chamber downstream of the pervious plate. This increases the speed of phase separation of the first contaminant, while still achieving proper mixing with the mixing means upstream of the pervious plate. As mentioned, the contents of the separation vessel may have a turbulent flow close to the mixing means, and the pervious plate may cause the flow to change into a laminar flow pattern, more suitable for allowing phase separation of the contaminant.

In another preferred embodiment, said mixing chamber is in the separation vessel, and wherein the settling chamber has a width larger than its height, and preferably the mixing chamber has a height larger than its width.

In other words, while the separation vessel may just comprise one vessel, the separation vessel may also comprise two interconnected chambers, i.e. a first or mixing chamber, connected to a second or settling chamber, downstream of the first chamber. The provision of a first and second chamber allows these chambers to be dimensioned in a way which improves the separation. The chosen dimensions of first chamber, in which the mixing means are preferably arranged, allow for proper mixing to occur between the phases present in the separation vessel, whereas the chosen dimensions of the second chamber allow for a more rapid formation of a contaminant layer with a certain thickness (as in size, not in consistency), which makes it easier to collect.

In a preferred embodiment, the separation vessel has a bottom which is elongate in at least one direction.

A bottom which is elongate in at least one direction, thereby giving the separation vessel the shape of a vessel with at least one elongate dimension, creates a large surface area for the phase separation to occur.

In another preferred embodiment, the separation vessel comprises a bottom outlet for collecting a second contaminant and/or a mixture comprising a second contaminant, said second contaminant being present in the feed stream.

### (Second Contaminant)

In addition to the first contaminant, the feed stream may also comprise a second contaminant, i.e. a contaminant which has a density higher than the main phase, which phase separates and/or precipitates below the main phase comprising the depolymerized condensation polymer in the separation vessel. Such second contaminants may for instance comprise sand or metals such as aluminum. In order to be able to collect this second contaminant phase, it is preferred to provide a collecting means for collecting this second contaminant. It is observed that a second contaminant may form a mixture and/or an agglomerate. Such agglomerate may be larger than only the second contaminant which was supplied as part of the feed stream. Such agglomerates may further comprise first contaminant and/or condensation polymer.

The aforementioned features have been found to provide an advantage in the collection of a first contaminant, may also provide an advantage for the collection of the second contaminant. This has in particular been found with the application of cooling, e.g. by introduction of water in the separation vessel, which may be followed by mixing and eventual passing through said pervious plate. An elongate bottom surface also contributed to a better separation of the second contaminant, because of the dependence of precipitation on gravity.

In a preferred embodiment, the separation vessel comprises at least one of an overflow baffle, for holding back the second contaminant and an underflow baffle, for holding back the first contaminant.

The provision of any of an overflow baffle and an underflow baffle has a number of advantages. In the first place, such baffles will in use extend into the contents of the separation vessel and are by that arrangement able to hold back a bottom and first contaminant layer, respectively. This can be useful in order to prevent a certain contaminant from entering a location (e.g. an outlet) downstream of the baffle, or to increase the ease of collection. Secondly, the provision of a baffle reduces the top surface available for forming a phase separated first contaminant layer without a substantial restriction in the volume of the separation vessel. As a consequence, the thickness of the layer (as in size, not in consistency) will increase, allowing for easier collection.

Underflow baffles typically suspends from the top of the separation vessel, e.g. from the ceiling thereof. Overflow baffles are typically mounted on the bottom of the separation vessel. Both types of baffles are typically arranged in the first half of the separation vessel in the main direction of flow.

More preferably, the reactor system comprises both an overflow and an underflow baffle. Preferably, the overflow baffle is arranged downstream of the underflow baffle in the main direction of flow in the separation vessel. Preferably, said baffles are arranged adjacent to each other in order to direct the flow of the contents of the separation vessel in a direction substantially perpendicular to the bottom of the separation vessel, thereby more effectively blocking any top and/or second contaminant from passing said baffles. Preferably, said baffles overlap in a direction perpendicular to the bottom of the separation vessel, thereby defining a channel between said baffles, which makes the blocking of any top and/or second contaminant even more effective. The baffles are preferably arranged in the first half of the separation vessel in the main direction of flow, especially when it is important to create a main phase buffer of a substantial volume, e.g. in a situation with a relatively large variation in the amount of input material.

In addition to eventual other mixing means, the reactor system may also comprise further mixing means to reduce the chance that any contaminants which have passed one or more baffles are able to settle.

In another preferred embodiment, the separation vessel further comprises a further separation means upstream of the depolymerization vessel, configured for separation of at least the condensation polymer from the feed stream and introduction thereof into the depolymerization vessel.

In some situations, e.g. when the feed stream comprises a relatively high amount of contaminants (e.g. more than 5% by weight, or maybe even more than 10% by weight), it may be preferred to carry out a pre-separation upstream of the depolymerization, which is carried out with a further separation means. The fraction of this separation which comprises the condensation polymer is subsequently transferred to the depolymerization vessel for depolymerization.

Such a further separation means may comprise any suitable separation means or combination of a number of separations. It is contemplated that such a further separation means at least comprises a washing or settling tank, in which the condensation polymer is mixed with a solvent. The solvent in this separate tank may be held at a temperature in a range between 30° C. and 70° C., more preferably between 35 and 55° C. The separation in this vessel is based on difference in density of the condensation polymer, the alcoholic solvent, the first contaminant (which will have a propensity to float in the solvent) and, where applicable, the second contaminant (which will have a propensity to sink in the solvent).

In yet another embodiment, the separation vessel is provided with a set of packed plates arranged in the separation vessel, for in use contacting the contents of the separation vessel.

These packed plates are designed to force the content of the separation vessel to lift over a certain distance, e.g. 5 centimeters. Any solid material, and fibers in particular, which will, when lifted accordingly, collide onto these packed plates will have an increased tendency and/or speed of settling, thereby increasing the ease and/or speed of separation. Fibers tend to be in particular present in waste streams comprising textiles, and are therefore in particular preferred when separating such streams. The provision of packed plates embodied as corrugated sheets is even more beneficial, since it increases the path length travelled by the contaminant, e.g. fiber, within the pack, thereby making the above effects occur in a more profound way.

### (Method)

As already disclosed above, the object of the invention is achieved with a method of separating a first contaminant from a feed stream further comprising a condensation polymer, which method comprises the steps of:
supplying the feed stream, an alcoholic solvent and optionally a depolymerization catalyst into a depolymerization vessel and mixing thereof to form a reaction mixture;
depolymerizing at least a portion of said condensation polymer in said reaction mixture into monomer, dimer, trimer and/or oligomer under said reaction conditions;
transferring the reaction mixture after the depolymerization of at least a portion of said condensation polymer in said reaction mixture to a separation stage comprising a separation vessel, and
collecting the first contaminant, wherein said first contaminant is separated from the alcoholic solvent on the basis of a density separation in the separation stage, in particular so that first contaminant is arranged on top of the alcoholic solvent in the separation vessel;
further comprising the step of cooling the reaction mixture with cooling means before the collecting step to ensure that the reaction mixture in the separation vessel is at a lower temperature than the depolymerization vessel such that the first contaminant that is liquid and/or dissolved in the depolymerization vessel at least partially precipitates and the cooled reaction mixture separates into a main phase that comprises the reaction products from the depolymerization and the alcoholic solvent, and a first contaminant phase in the form of solid particles and/or a solid layer.

In an embodiment, the collecting step further comprises transferring the reaction mixture from the separation vessel to a sieve bend unit arranged downstream of the separation vessel for separating the first contaminant from a filtrate stream comprising the alcoholic solvent via an inclined screen. Preferably, the filtrate stream is at least partly recirculated to the separation vessel.

In an embodiment, the reaction mixture is separated by at least one cyclone device arranged downstream of the depolymerization vessel into a low density stream comprising the first contaminant and a high density stream comprising the alcoholic solvent on the basis of a density separation. In particular, a filter device is arranged for receiving at least one low density stream from said at least one cyclone device to filter the first contaminant from the alcoholic solvent.

Typically, the depolymerization involves heating of the condensation polymer. The condensation polymer may be heated in a variety of ways. It may for instance be heated with heating means present in the depolymerization vessel, but it is also possible to heat the stream by the introduction of a heated solvent into the depolymerization, which solvent will thereby heat the stream.

Examples of suitable catalysts are mentioned in the published patent applications WO 2018/143798 A1, WO 2017/111602 A1, WO 2016/105200 A1 and WO 2014/209117 A1, all filed by the applicant. Other catalysts may also be considered.

The condensation polymer is more preferably one of a polyester, polyamide, polyurethane and polyether, the latter also including starch and cellulose based polymers. Polyesters are preferred, and polyethylene terephthalate (PET) is currently commercially the most important polyester. PET may include further comonomers, such as iso-BHET, to improve its properties, as known in the art. Other polyesters are however not excluded. Examples include so-called biodegradable polymers, such as polylactic acid (PLA), polybutylene terephthalate (PBT), polycyclohexylenedimethylene-2,5-furandicarboxylate (PCF), polybutylene adipate-co-terephthalate (PBAT), polybutylene sebacate-co-terephthalate (PBSeT), polybutylene succinate-co terephthalate (PBST), polybutylene 2,5 furandicarboxylate-co-succinate (PBSF), polybutylene 2,5-firandicarboxylate-co-adipate (PBAF), polybutylene 2,5-furandicarboxylate-co-azelate (PBAzF), polybutylene 2,5 furandicarboxylate-co-sebacate (PBSeF), polybutylene 2,5-furandicarboxylate-co-brassylate (PBBrF), polybutylene 2,5-furandicarboxylate (PBF), polybutylene succinate (PBS), polybutylene adipate (PBA), polybutylene succinate-co-adipate (PBSA), polybutylene succinate-co-sebacate (PBSSe), polybutylene sebacate (PBSe), and copolymers thereof, for instance copolymers with polylactic acid and/or PET.

In a preferred implementation, the stream is substantially dry, and more particularly has a water content as low as reasonably possible, for instance less than 5 wt %, preferably less than 3 wt %, more preferably less than 1 wt %.

In a preferred embodiment of the method, the separation vessel is provided with a top outlet, of which the position is adjustable and the method comprises the steps of:
detecting the location of the contaminant;
adjusting the position of the top outlet for collecting the first contaminant.

The adjustability of the position of the top outlet allows for a number of strategies in using the top outlet.

In an embodiment of the method, the position of the outlet is controlled, e.g. electronically, to ensure that the top outlet is always in contact with the top of the contents of the separation vessel. In other words, a rise or fall in the contents level inside the separation vessel will lead to an adjustment of the position of the top outlet to accommodate for this rise or fall.

In another embodiment of the method, the outlet generally does not contact the constituents of the separation vessel. In this situation, the first contaminant layer will build up or grow over time. After a certain suitable period of time, the top outlet is then, from its default position, brought into a position for collecting the first contaminant and, thereafter returned to its default position.

In a preferred embodiment of the method, the method comprises the step of cooling the reaction mixture with cooling means to ensure that the separation vessel is at a lower temperature than the depolymerization vessel, preferably such that a contaminant that is liquid and/or dissolved in the depolymerization vessel forms a separate phase and/or at least partially precipitates.

In another preferred embodiment of the method, the method further comprises the step of introducing water into the separation vessel.

Again, the water added may be impure water and/or aqueous solutions, e.g. solutions which consist of more than 85 percent by weight of water, maybe even more than 90 percent by weight of water, or maybe even more than 95 percent by weight of water. It may be preferred to use a residual aqueous solution which may originate from another process.

In a further preferred embodiment of the method, the method further comprises the step of mixing the contents of the separation vessel with mixing means.

In yet a further preferred embodiment of the method, the method further comprises the step of passing the mixed contents of the separation vessel through a pervious plate, which is arranged in the separation vessel downstream of and preferably adjacent to the mixing means, so as to define a mixing chamber in and/or upstream of the separation vessel and a settling chamber, downstream of the mixing chamber.

In a preferred embodiment of the method, the method further comprises the step of collecting a second contaminant or a mixture comprising a second contaminant with a bottom outlet, wherein the second contaminant is supplied as part of the feed stream.

In a preferred embodiment of the method, the method further comprises the step of holding back the top and/or second contaminant with a respective underflow and/or overflow baffle, arranged in the separation vessel.

In a preferred embodiment of the method, the contaminant comprises a polyolefin.

Polyolefins are a common ingredient of waste streams, and are therefore an attractive candidate for separation from streams comprising condensation polymers.

In a preferred embodiment of the method, the contaminant comprises a pigment, preferably a blue pigment.

Pigments may be included in the condensation polymer and/or any polyolefin present in the feed stream. Upon disintegration of the condensation polymer, such pigments are liberated and may dissolve into the alcoholic solvent or may not dissolve therein but rather attach to solid material. It has been observed, surprisingly that some pigments tend to separate out of the reaction mixture with the first contaminant, which is particularly polyolefin. Without restricting the scope of protection, it is suspected that such contaminants are non-polar to such an extent that they do not match the polarity of the alcoholic solvent. This allows the polyolefins to reduce the pigment concentration in the reaction mixture, which may thereafter be subjected to post-processing, e.g. treatment with active carbon. As a consequence, the post-processing equipment may be implemented in a smaller design or may even be omitted. For that reason, when dealing with the separation of feed streams comprising a pigment, it may be beneficial to in addition also incorporate a minimum amount of polyolefin, e.g. at least 1% by weight of the composition, or even at least 2% by weight of the composition. This is in stark contrast with the current view on separation of waste streams, in which it is preferred to keep the amount of contaminants in the starting material as low as possible.

In particular, it has been found that the above separation of pigments together with polyolefins is pronounced when the pigment is a blue pigment, e.g. phthalocyanine.

In a preferred embodiment of the method, the condensation polymer is a polyester, more preferably polyethylene terephthalate.

In a preferred embodiment of the method, the stream comprises waste material in solid form, preferably in fragments, such as flakes.

Waste material in solid form allows the stream to be easily processed. It is preferred to introduce the waste material in the form of flakes. It increases the rate of depolymerization, and makes it easier to introduce the stream into the depolymerization vessel. Flakes for instance have a volume of between 5.10-6 and 0.5 cm3, more preferably of between 5.10-4 and 0.05 cm3. If the feed stream would be provided in larger sizes, a size reduction step may be carried out, for instance by shredding and/or grinding.

In a preferred embodiment of the method, the step of bringing the reaction mixture under said reaction conditions comprises the step of heating the reaction mixture to a temperature of between 170° C. and 200° C.

The temperature in the depolymerization vessel is preferably in the range of 170-200° C. for depolymerization of polyester and more particularly PET.

In a preferred embodiment of the method, the step of depolymerizing is substantially by glycolysis.

Glycolysis is a known process for converting condensation polymers, and polyethylene terephthalate in particular, into bis(2-hydroxyethyl)terephthalate (BHET) and oligomers in a transesterification, not requiring expensive distillation. Such BHET is, where applicable after eventual post-processing, considered a virgin quality material that can be used as a starting material for making new polyethylene terephthalate.

In a preferred embodiment of the method, the solvent comprises an alcoholic solvent, such as ethylene glycol.

Most effective temperatures for depolymerization are in the range of 190-200° C., in combination with the use of ethylene glycol as a solvent. The temperature for dissolution of PET into the solvent such as ethylene glycol could be achieved in the range of 120-180° C., for instance 150-180° C.

According to again a further aspect, the invention relates to a method of processing a feed stream comprising a condensation polymer and a first contaminant. Said processing comprises separating the first contaminant according to the invention, and post-processing remaining reaction mixture into purified monomer and/or purified dimer. The latter post-processing preferably occurs by crystallization of the dimer and/or the monomer. Such may be effected as described in the non-pre-published applications NL2023681 and NL2023686, which are included herein by reference.

According to again a further aspect, processing of a feed stream comprising condensation polymer and a first contaminant is performed by using the reactor system in accordance with the invention. Preferably, the processing further comprises purifying of the dimer and/or monomer so as to arrive at products that are suitable for polymerization reactions.

It is observed for clarity that any embodiment or implementation discussed hereinabove is applicable to any of the aspects (e.g. reactor system, method) covered in the present application. Consequently, the hereinabove described methods are preferably carried out in the hereinabove described reactor systems.

### BRIEF INTRODUCTION OF THE FIGURES

These and other aspects of the method and the reactor system of the invention will be further elucidated with reference to the figures, which are purely diagrammatical in nature and not drawn to scale, wherein:

FIG. 1 shows a schematic diagram of embodiment of the reactor system according to the invention;

FIG. 2 shows a first embodiment of a separation vessel in for the reactor system according to FIG. 1;

FIG. 3 shows a second embodiment of a separation vessel in for the reactor system according to FIG. 1;

FIG. 4 shows a third embodiment of a separation vessel in for the reactor system according to FIG. 1;

FIG. 5 shows a fourth embodiment of a separation vessel in for the reactor system according to FIG. 1;

FIG. 6 shows a fifth embodiment of a separation vessel in for the reactor system according to FIG. 1;

FIG. 7 shows an embodiment of a further separation means for the reactor system according to FIG. 1;

FIG. 8 shows an embodiment of a method according to the invention;

FIG. 9 shows a further embodiment of the reactor system according to the invention comprising a separation stage including a sieve bend unit;

FIG. 10 shows a further embodiment of the reactor system according to the invention comprising a separation stage including a number of cyclones;

FIG. 11 shows a further embodiment of the reactor system according to the invention comprising a separation stage including a sieve bend unit.

### DETAILED DESCRIPTION OF ILLUSTRATED EMBODIMENTS

In the following, equal or corresponding parts in different figures will be referred to with equal reference numerals. The illustrated embodiments are intended for explanation and illustration and are not intended to limit the scope of the claims.

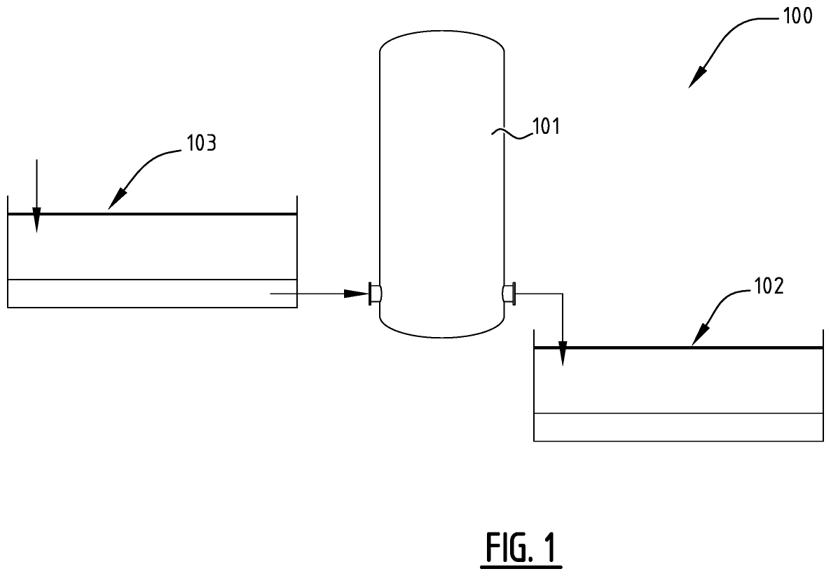

In FIG. 1, a schematic diagram of an embodiment of a reactor system 100 according to the invention is disclosed. The reactor system 100 comprises a depolymerization vessel 101 for depolymerizing a condensation polymer in a stream further comprising a contaminant such as a waste stream, and a separation vessel 102, downstream of the depolymerization vessel 101. Optionally, the reactor system further comprises a further separation means 103, upstream of the depolymerization vessel.

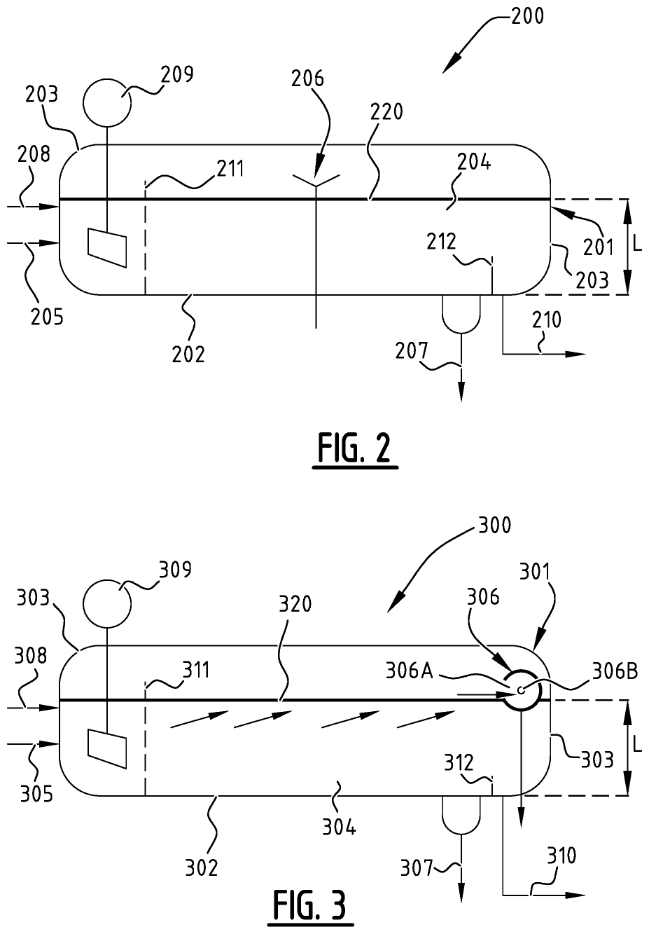

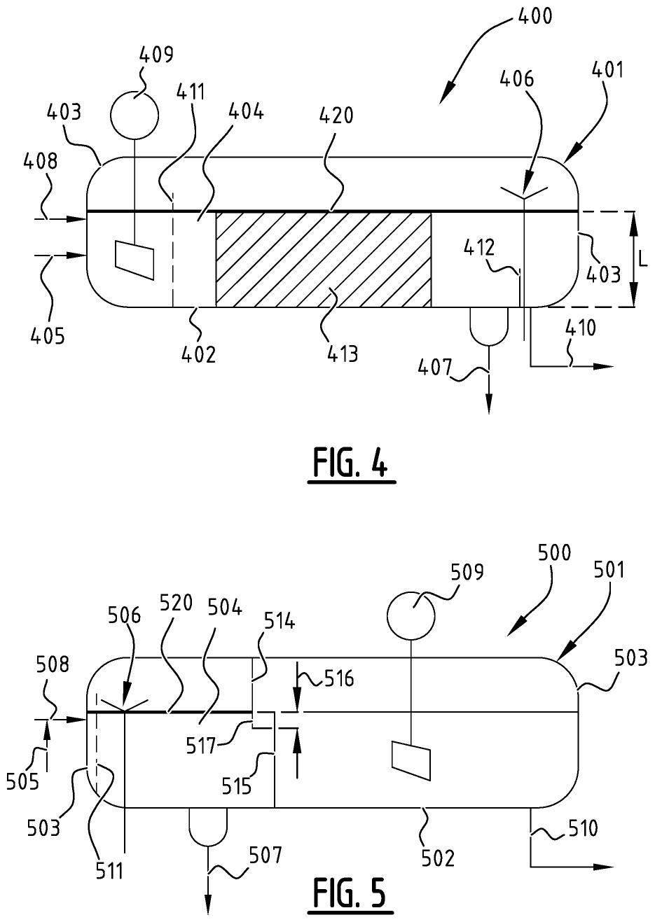

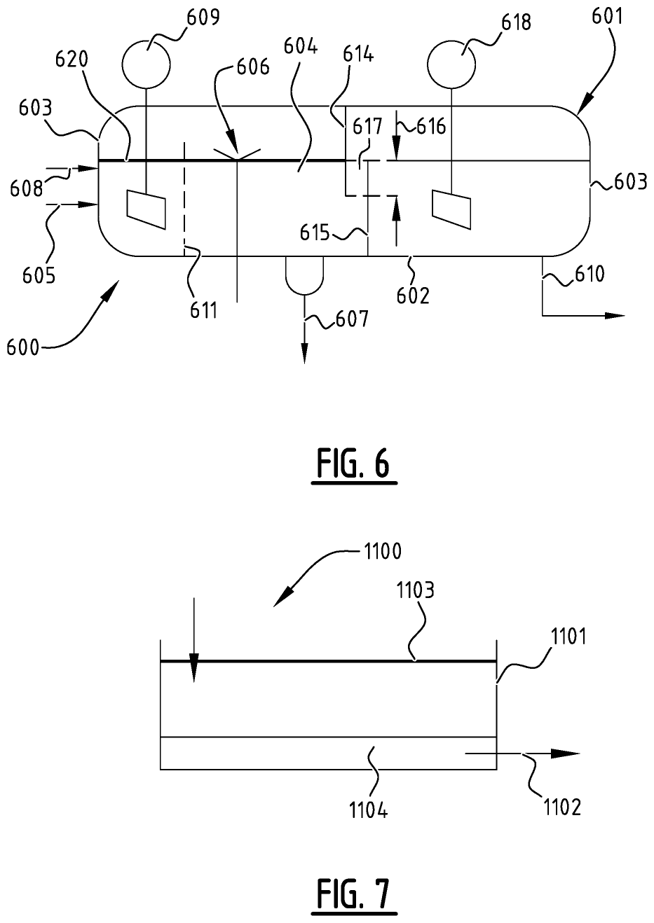

The depolymerization vessel 101 may be any vessel considered suitable for the intended purpose as described in the preceding sections. The separation vessel 102 may be embodied in a number of ways, of which a few examples 200, 300, 400, 500 are disclosed in FIGS. 2 to 6.

In each of the embodiments, the embodiment 200, 300, 400, 500, 600 comprises a separation vessel 201, 301, 401, 501, 601 with a bottom 202, 302, 402, 502, 602 and side walls 203, 303, 403, 503, 603, and which is in use filled with a mixture 204, 304, 404, 504, 604 up to a level L. In each of the embodiments 200, 300, 400, 500, 600, the separation vessel 201, 301, 401, 501, 600 extends from an inlet 205, 305, 405, 505, 605 downstream of the depolymerization vessel 101 towards a number of outlets, which in these cases comprise a skimmer 206, 406, 506, 606 or an adjustable scum pipe 306, each for collecting a first contaminant 220, 320, 420, 520, 620 in the mixture 204, 304, 404, 504, 604, i.e. a contaminant which has a density lower than the depolymerized condensation polymer in the mixture 204, 304, 404, 504, 604, and which will float on the solvent and a bottom outlet 207, 307, 407, 507, 607, for collecting a second contaminant, i.e. a contaminant which has a density higher than the depolymerized condensation polymer in the mixture 204, 304, 404, 504, 604. In each of the embodiments 200, 300, 400, 500, 600, the separation vessel 201, 301, 401, 501, 601 is further provided with a further inlet 208, 308, 408, 508, 608, which is connected to a water supply, and which is used to introduce water into the separation vessel 201, 301, 401, 501, 601 and thereby cools the mixture 204, 304, 404, 504, 604 to a temperature lower than in the depolymerization vessel 101 such that at least contaminant that is liquid and/or dissolved in the depolymerization vessel 101 forms a separate phase and/or at least partially precipitates in the separation vessel 201, 301, 401, 501, 601. In each of the embodiments 200, 300, 400, 500, 600, the separation vessel 201, 301, 401, 501, 601 is further provided with mixing means for mixing the reaction mixture originating from the depolymerization vessel 101 with the water (or other aqueous solution) originating from the water supply, introduced through further inlet 208, 308, 408, 508, 608. In some embodiments, the mixing means comprise a mixer 209, 309, 409, 609 arranged in the separation vessel 201, 301, 401, 601, downstream of the inlet 205, 305, 405, 605. In another embodiment, the mixing means are arranged in the supply lines 505, 508 to the separation vessel 501, and may be embodied as an inline mixer. In each of the embodiments 200, 300, 400, 500, 600, the separation vessel 201, 301, 401, 501, 601 is further provided with a discharge outlet 210, 310, 410, 510, 610 for discharging the depolymerized condensation polymer, e.g. to a post-processing unit.

In the separation vessel 201, 301, 401, 501, 601, the contaminant in the mixture 204, 304, 404, 504, 604 that is liquid and/or dissolved in the depolymerization vessel, which is introduced through the inlet 205, 305, 405, 505, 605, originating from the depolymerization vessel 101, and which further comprises an at least partially depolymerized condensation polymer and a solvent, will form a separate phase and/or at least partially precipitates in the separation vessel 201, 301, 401, 501, 601.

It is important to emphasize that the features which are common to each of the previous embodiments are not necessary in order to obtain the effects of the invention.

In the embodiments 200, 300, 400, 500 and 600, the separation vessel is provided with a pervious plate 211, 311, 411, 511, 611, arranged in the separation vessel 201, 301, 401, 501, 601, for settling the mixture 204, 304, 404, 504, 604, downstream of and adjacent to the mixing means 209, 309, 409, 505; 508, 609.

In the embodiments 200, 300 and 400, the discharge outlet 210, 310, 410 is provided with an upright baffle 212, 312, 412 arranged on the bottom 202, 302, 402 of the separation vessel 201, 301, 401 upstream of the discharge outlet 210, 310, 410, in order to prevent any contaminants, precipitating contaminants in particular, from entering the discharge outlet 210, 310, 410.

In the embodiments 200, 300, 400 and 600, the or part of the mixing means 209, 309, 409, 609 are arranged downstream of and adjacent to the inlet 205, 305, 405, 609 and the further inlet 208, 308, 408, 608, whereas in the embodiment 500, the mixing means 509 are arranged within the supply line 505; 508.

In the second embodiment 300, the position of the opening 306a of the scum pipe 306 may be changed by rotation of the scum pipe 306 around its axis 306b, in order to accommodate for changes in the fluid level L.

In the third embodiment 400, a set of packed plates 413 is arranged between the pervious plate 411 and the top outlet 406 (which is moved independent of the position of outlets 407, 410 to a location further downstream of the inlet 405 in order to create space for the set of packed plates 413). These packed plates 413 lift the mixture 404 in the separation vessel 401 and thereby make the contaminants collide onto the plates, thereby increase the ease and speed of separation of the contaminants from the mixture 404.

In the fourth and fifth embodiments 500, 600, the separation vessels 501, 601 are furthermore provided with an underflow baffle 514, 614 for holding back a contaminant which has a density lower than the depolymerized condensation polymer and an overflow baffle 515, 615, downstream of the underflow baffle 514, 614, for holding back a second contaminant. The baffles 514; 515, 614; 615 are arranged in the first half of the separation vessel 501, 601 in the main direction of flow, i.e. from inlets 505, 605 towards discharge outlets 510; 610, and are arranged adjacent to each other in order to direct the flow of the mixture 504, 604 in a direction substantially perpendicular to the bottom 502, 602 of the separation vessels 501, 601, overlapping in regions 516, 616 defining a channel 517, 617 in between the walls 514; 515, 614; 615, defining a volume within the separation vessel 501, 601 for building a main phase buffer downstream of the baffles 514; 515, 614; 615.

The fifth embodiment 600 further comprises an optional further mixing means 618 for mixing the mixture 604 downstream of the baffles 614; 615, to reduce the chance that any contaminants which have passed baffles 614; 615 are able to settle.

A possible embodiment 1100 of a further separation means 103 is disclosed in FIG. 7. This separation means 1100 comprises a separation vessel 1101 with an open top and an outlet 1102, connected to the inlet of depolymerization vessel 101. A stream may be introduced from the top of the separation vessel 1101 and dissolved in an alcoholic solvent, such as ethylene glycol. Floating material 1103 may be removed from the open top, and the bottom fraction 1104, which typically comprises most of the condensation polymer flakes, is transferred to the depolymerization vessel 101 for depolymerization.

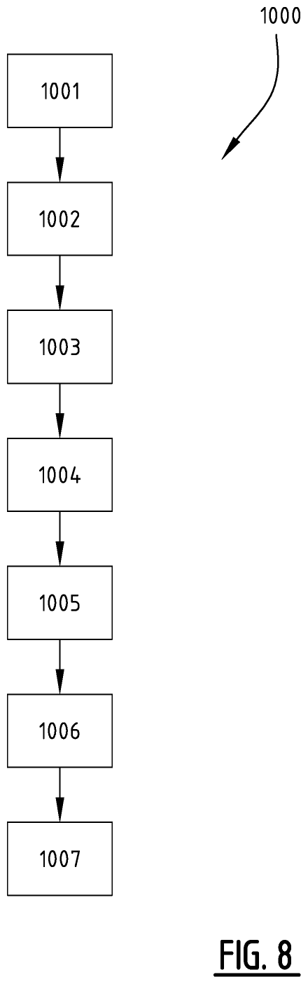

An embodiment 1000 of a method according to the invention, disclosed in FIG. 8, comprises:

the step 1001 of bringing the stream which constitutes a reaction mixture that further comprises a solvent, selected to be a solvent for the condensation polymer and/or for reaction products obtained from said condensation polymer by depolymerization, and optionally a catalyst under said reaction conditions in a depolymerization vessel 101;

the step 1002 of depolymerizing at least a portion of said condensation polymer in said reaction mixture into monomer, dimer, trimer and/or oligomer under said reaction conditions;

the step 1003 of transferring the reaction mixture after the depolymerization of at least a portion of said condensation polymer in said reaction mixture to a separation vessel 201, 301, 401, 501, 601 through the inlet 205, 305, 405, 505, 605 thereof;

the step 1004 of cooling the reaction mixture by the introduction of water via a further inlet 208, 308, 408, 508, 608 to ensure that the separation vessel 201, 301, 401, 501, 601 is at a lower temperature than the depolymerization vessel 101, and such that the contaminant that is liquid and/or dissolved in the depolymerization vessel 101 forms a separate phase and/or at least partially precipitates;

the step 1005 of mixing the reaction mixture with the water introduced into the separation vessel with mixing means 209, 309, 409, 609 arranged in the separation vessel 201, 301, 401, 601, downstream of to the inlet 205, 305, 405, 505, 605 and the further inlet 208, 308, 408, 508, 608;

the step 1006 of collecting a first contaminant with the top outlet based on density separation in the separation vessel, and

the step 1007 of discharging the depolymerized condensation polymer from the separation vessel 201, 301, 401, 501, 601.

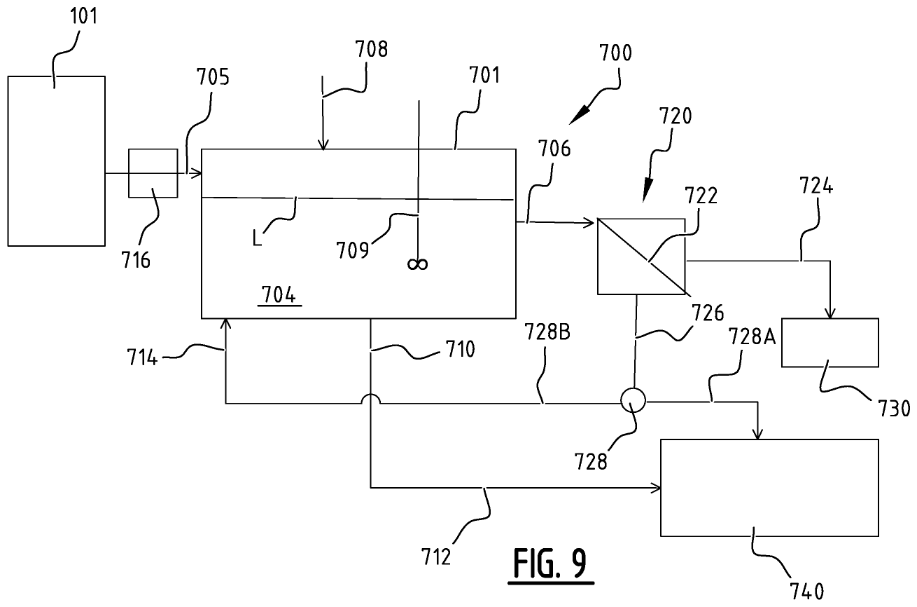

FIG. 9 shows a further embodiment of the reactor system according to the invention comprising a separation stage including a sieve bend unit. The reactor system comprises the depolymerization vessel 101 for depolymerizing a condensation polymer in a stream further comprising a contaminant such as a waste stream. The reactor system further comprises a separation stage 700, which comprises a separation vessel 701 and a sieve bend unit 720. The separation stage 700 is arranged downstream of the depolymerization vessel 101. A stream of reaction mixture is fed to the separation vessel 701 via inlet 705.

Optionally, a heat exchanger is arranged between the depolymerization vessel 101 and the separation vessel 701 to cool the reaction mixture such that the first contaminant forms a separate phase having solid first contaminant. In particular, the reaction mixture may separate into a first contaminant phase comprising the first contaminant and a main phase, which predominantly comprises other components, such as the alcoholic solvent. The solid first contaminant has a density lower than the depolymerized condensation polymer and the alcoholic solvent in the reaction mixture.

Alternatively or additionally, water is introduced into the separation vessel 701 in order to cool the reaction mixture 704 in the separation vessel 701 such that the first contaminant may form a separate phase having solid first contaminant.

In particular, the reaction mixture separates into a first contaminant phase comprising the first contaminant having a solid state and a main phase, which predominantly comprises other components, such as the alcoholic solvent. The first contaminant in the solid state has a density lower than the depolymerized condensation polymer and the alcoholic solvent in the reaction mixture.

In particular, the reaction mixture 704 is cooled such that first contaminant at least partially precipitates to form a solid phase in the form of solid particles and/or a solid layer.

The separation vessel 701 further comprises mixing means 709 to mix the reaction mixture 704 to enhance the cooling of the reaction mixture 704 after adding water via the water inlet 708. The reaction mixture 704 may be present in the separation vessel 701 up to a liquid surface level L.

The separation vessel 701 further comprises a top outlet 706 and a bottom outlet 710. The top outlet 706 is arranged at a level to carry off the first contaminant phase. The first contaminant phase is transferred to the sieve bend unit 720. The sieve bend unit 720 comprises a screen 722 for separating the first contaminant 724 from a filtrate stream 726 comprising the alcoholic solvent. The filtrate stream 726 may in particularly comprise other constituents than the first contaminant, such as the alcoholic solvent and the depolymerized condensation polymer. In this embodiment, the sieve bend unit is coupled to the top outlet of the separation vessel for receiving the first contaminant phase. The screen is an inclined screen 722. The screen is arranged inclined to allow the residue 724 comprising the solid first contaminant part to slide downwards along the inclined sieve bend 722 towards a storage vessel 730. The storage vessel 730 is arranged for storing the solid first contaminant part, which falls due to gravity into the storage vessel 730 via a chute.

The filtrate stream 726 may be selectively guided by the valve 728 in a product stream 728A to the post-processing vessel 740 and/or may be at least partly recirculated to the separation vessel 701 in a recirculation stream 728B. Additionally, the bottom outlet 710 is arranged at a level to carry off the main phase 712 towards to the post-processing vessel 740.

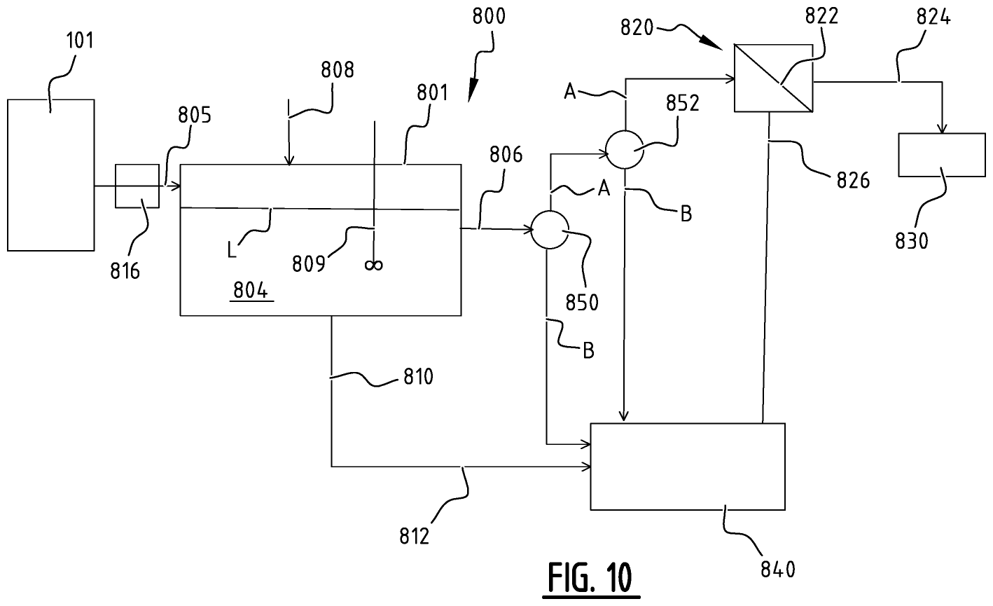

FIG. 10 shows a further embodiment of the reactor system according to the invention comprising a separation stage including a number of cyclones. The reactor system comprises the depolymerization vessel 101. The reactor system further comprises a separation stage 800, which comprises a separation vessel 801 and a number of cyclones 850, 852. The separation stage 800 is arranged downstream of the depolymerization vessel 101. The separation vessel 801 comprises an inlet 805, a top outlet 806, a water inlet 808, a bottom outlet 810, mixing means 809 and holds the reaction mixture 804 in a same way is shown in the embodiment of FIG. 9. Optional a heat exchanger 816 as cooling means is arranged between the depolymerization vessel 101 and the separation vessel 801 to cool the reaction mixture 804 such that the first contaminant may form a separate phase having solid first contaminant.

The top outlet 806 is arranged at a level to carry off the first contaminant phase to the first cyclone 850. The first contaminant phase is transferred to the first cyclone 850, where it is separated into a low density stream A comprising the first contaminant and a high density stream B comprising the alcoholic solvent on the basis of a density separation. The low density stream A is transferred to a second cyclone 852, where the low density stream A is further separated into a low density stream A comprising the first contaminant and a high density stream B comprising the alcoholic solvent on the basis of a density separation. The high density stream B of the first cyclone 850 and the high density stream B of the second cyclone 852 are transferred to a post-processing vessel 840. The low density stream A of the second cyclone 852 is transferred to a filter device 820, such as a sieve bend unit or any other filter unit, for separating the, solid, first contaminant 824 from the liquid phase of the low density stream A. The liquid phase 826, which comprises the alcoholic solvent and/or the depolymerized condensation polymer components, is transferred to the post-processing vessel 840. The solid first contaminant 824, being the residue of the filter device 820 is collected in a storage vessel 830, e.g. by allowing the solid first contaminant 824 to fall due to gravity into the storage vessel 830 via a chute.

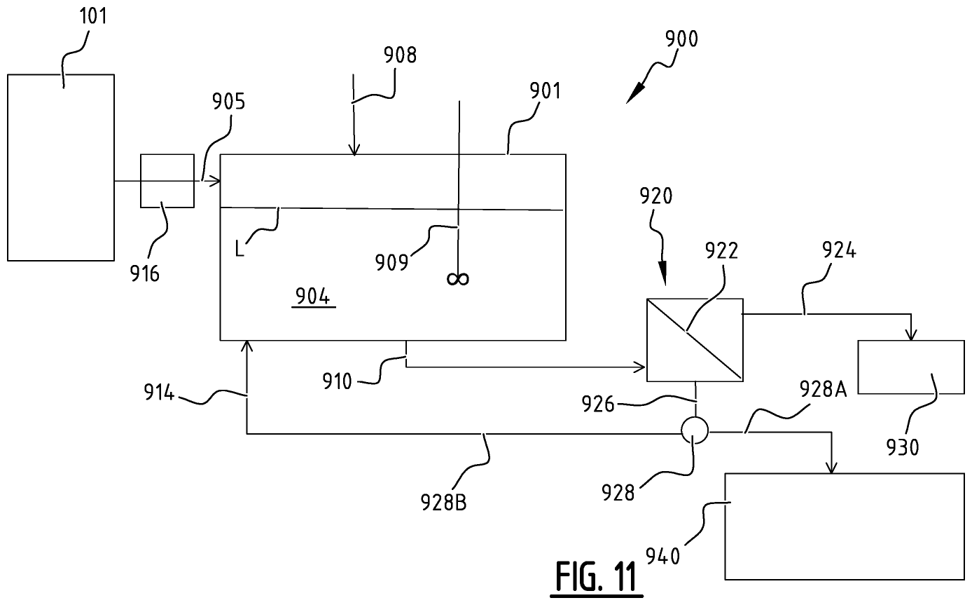

FIG. 11 shows a further embodiment of the reactor system according to the invention comprising a separation stage including a sieve bend unit. The embodiment is a modified embodiment compared to the embodiment shown in FIG. 9. The separation vessel 901 comprises an inlet 905, a water inlet 908, a bottom outlet 910, mixing means 909 and holds the reaction mixture 904 in a same way is shown in the embodiment of FIG. 9. In the embodiment of FIG. 11, the bottom outlet 910 is arranged to carry off the reaction mixture 904, preferably the whole contents, of the separation vessel 901, including a first contaminant phase and a main phase, to the sieve bend unit 920. The reaction mixture is processed by the sieve bend unit 920 in a same way as sieve bend unit 720, thereby the first contaminant 924 from a filtrate stream 926 comprising the alcoholic solvent. The sieve bend unit 920 is similar to a sieve bend unit 720, i.e. having an inclined screen 922. The screen is arranged inclined to allow the residue 924 comprising the solid first contaminant part to slide downwards along the inclined sieve bend 922 towards a storage vessel 930.

The filtrate stream 926 may be selectively guided by the valve 928 in a product stream 928A to the post-processing vessel 940 and/or may be at least partly recirculated to the separation vessel 901 in a recirculation stream 928B.

The embodiment has the advantage that the whole content 904 of the separation vessel 901 is processed by the sieve bend unit 920. The main phase, which may be predominantly disposed below the first contaminant phase due to density separation in the separation vessel 901, will predominantly be processed first by the sieve bend unit 920 prior to the first contaminant phase. This has the advantage of an efficient and fast separation of the filtrate stream 926 from the first contaminant 924.

In all of the embodiments, the inclined screen 722, 922 preferably has a plurality of slits, each having a slit spanning dimension in the range of 250-500 μm. The longitudinal direction of the slits is arranged substantially perpendicular to a feed direction of the material over the inclined screen.

In an even further embodiment, the embodiment of FIG. 10 having a separation stage 800, comprising a separation vessel 801 and a number of cyclones 850, 852, is modified by connecting the bottom outlet 810 to the number of cyclones 850, 852 to process the whole contents 804 of the separation vessel 801 by the cyclones 850, 852 and by the filter device 820 in a similar way is described for the embodiment shown in FIG. 10 for the first contaminant phase.

1-40. (canceled)

41. Method of separating a first contaminant from a feed stream further comprising a condensation polymer, which method comprises the steps of:
supplying the feed stream, an alcoholic solvent and optionally a depolymerization catalyst into a depolymerization vessel and mixing thereof to form a reaction mixture;
depolymerizing at least a portion of said condensation polymer in said reaction mixture into monomer, dimer, trimer and/or oligomer under said reaction conditions;
transferring the reaction mixture after the depolymerization of at least a portion of said condensation polymer in said reaction mixture to a separation stage comprising a separation vessel, and
collecting the first contaminant, wherein said first contaminant is separated from the alcoholic solvent on the basis of a density separation in the separation stage, in particular so that first contaminant is arranged on top of the alcoholic solvent in the separation vessel;
further comprising the step of cooling the reaction mixture with cooling means before the collecting step to ensure that the reaction mixture in the separation vessel is at a lower temperature than the depolymerization vessel such that the first contaminant that is liquid and/or dissolved in the depolymerization vessel at least partially precipitates and the cooled reaction mixture separates into a main phase that comprises the reaction products from the depolymerization and the alcoholic solvent, and a first contaminant phase in the form of solid particles and/or a solid layer.

42. Method of claim 41, wherein the separation vessel is provided with a top outlet, of which the position is adjustable and wherein the method comprises the steps of:
detecting the location of the first contaminant;
adjusting the position of the top outlet for collecting the first contaminant.

43. Method of claim 41, further comprising the step of introducing water into the separation vessel, wherein optional the water provides the cooling means for the step of cooling the reaction mixture.

44. Method of claim 41, wherein the collecting step further comprises transferring the reaction mixture from the separation vessel to a sieve bend unit arranged downstream of the separation vessel for separating the first contaminant from a filtrate stream comprising the alcoholic solvent via an inclined screen.

45. Method of claim 44, wherein the filtrate stream is at least partly recirculated to the separation vessel.

46. Method of claim 41, wherein the reaction mixture is separated by at least one cyclone device arranged downstream of the depolymerization vessel into a low-density stream comprising the first contaminant and a high-density stream comprising the alcoholic solvent on the basis of a density separation.

47. Method of claim 41, wherein the reaction mixture is separated by at least one cyclone device arranged downstream of the separation vessel into a low-density stream comprising the first contaminant and a high-density stream comprising the alcoholic solvent on the basis of a density separation.

48. Method of claim 46, wherein a filter device is arranged for receiving at least one low density stream from said at least one cyclone device to filter the first contaminant from the alcoholic solvent.

49. Method of claim 41, further comprising the step of collecting a second contaminant or a mixture comprising a second contaminant with a bottom outlet, wherein the second contaminant is supplied as part of the feed stream.

50. Method of claim 41, further comprising the step of holding back the top and/or second contaminant with a respective underflow and/or overflow baffle, arranged in the separation vessel.

51. Method of claim 41, wherein the first contaminant comprises or is a polyolefin, optionally further comprising a pigment, preferably a blue pigment.

52. Method of claim 41, wherein the condensation polymer is a polyester, more preferably polyethylene terephthalate.

53. Method of claim 41, wherein the step of bringing the reaction mixture under said reaction conditions comprises the step of heating the reaction mixture to a temperature of between 170° C. and 200° C.

54. Method of claim 53, wherein the separation vessel is cooled to a temperature that is between 10° C. and 110° C. lower than the temperature to which the reaction mixture is heated.

55. Method of claim 41, wherein the step of depolymerizing is by glycolysis, and the alcoholic solvent is an alkanediol, such as ethylene glycol.

56. A reactor system for carrying out the method in accordance with claim 41, the reactor system comprising:
at least one depolymerization vessel, configured for depolymerizing a condensation polymer into monomer, dimer, trimer and/or oligomer, which depolymerizing occurs in an alcoholic solvent, wherein said condensation polymer is provided as a feed stream further comprising a first contaminant,
a separation stage, downstream of the depolymerization vessel, configured for collecting the first contaminant, said separation stage comprising a separation vessel and
optionally a sieve bend unit arranged downstream of the separation vessel for separating the first contaminant from a filtrate stream comprising the alcoholic solvent via an inclined screen, and/or one or more cyclone devices,
which separation vessel has an inlet for introducing the reaction mixture originating from the depolymerization vessel into the separation vessel, in which separation vessel said first contaminant is separated from the alcoholic solvent on the basis of a density separation so that first contaminant is arranged on top of the alcoholic solvent,
wherein the reactor system further comprises a cooling means for ensuring that the separation vessel is at a lower temperature than the depolymerization vessel such that the first contaminant that is liquid and/or dissolved in the depolymerization vessel at least partially precipitates and the cooled reaction mixture separates into a main phase that comprises the reaction products from the depolymerization and the alcoholic solvent, and a first contaminant phase in the form of solid particles and/or a solid layer.
